SEQUENCE LISTING (2 sequences)

SEQ ID NO: 1 - protein, 194 aa, Homo sapiens
MHKWILTWILPTLLYRSCFHIICLVGTISLACNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAIT

SEQ ID NO: 2 - DNA, 3936 bp, Homo sapiens
agttttaattgcttccaatgaggtcagcaaaggtatttatcgaaaagccctgaataaaaggctcacacacacacacaagcacacacgcgctcacacacagagagaaaatccttctgcctgttgatttatggaaacaattatgattctgctggagaacttttcagctgagaaatagtttgtagctacagtagaaaggctcaagttgcaccaggcagacaacagacatggaattcttatatatccagctgttagcaacaaaacaaaagtcaaatagcaaacagcgtcacagcaactgaacttactacgaactgtttttatgaggatttatcaacagagttatttaaggaggaatcctgtgttgttatcaggaactaaaaggataaggctaacaatttggaaagagcaactactctttcttaaatcaatctacaattcacagataggaagaggtcaatgacctaggagtaacaatcaactcaagattcattttcattatgttattcatgaacacccggagcactacactataatgcacaaatggatactgacatggatcctgccaactttgctctacagatcatgctttcacattatctgtctagtgggtactatatctttagcttgcaatgacatgactccagagcaaatggctacaaatgtgaactgttccagccctgagcgacacacaagaagttatgattacatggaaggaggggatataagagtgagaagactcttctgtcgaacacagtggtacctgaggatcgataaaagaggcaaagtaaaagggacccaagagatgaagaataattacaatatcatggaaatcaggacagtggcagttggaattgtggcaatcaaaggggtggaaagtgaattctatcttgcaatgaacaaggaaggaaaactctatgcaaagaaagaatgcaatgaagattgtaacttcaaagaactaattctggaaaaccattacaacacatatgcatcagctaaatggacacacaacggaggggaaatgtttgttgccttaaatcaaaaggggattcctgtaagaggaaaaaaaacgaagaaagaacaaaaaacagcccactttcttcctatggcaataacttaattgcatatggtatataaagaaccagttccagcagggagatttctttaagtggactgttttctttcttctcaaaattttctttccttttattttttagtaatcaagaaaggctggaaaactactgaaaaactgatcaagctggacttgtgcatttatgtttgttttaagacactgcattaaagaaagatttgaaaagtatacacaaaaatcagatttagtaactaaaggttgtaaaaaattgtaaaactggttgtacaatcatgatgttagtaacagtaatttttttcttaaattaatttacccttaagagtatgttagatttgattatctgataatgattatttaaatattcctatctgcttataaaatggctgctataataataataatacagatgttgttatataaggtatatcagacctacaggcttctggcaggatttgtcagataatcaagccacactaactatggaaaatgagcagcattttaaatgctttctagtgaaaaattataatctacttaaactctaatcagaaaaaaaattctcaaaaaaactattatgaaagtcaataaaatagataatttaacaaaagtacaggattagaacatgcttatacctataaataagaacaaaatttctaatgctgctcaagtggaaagggtattgctaaaaggatgtttccaaaaatcttgtatataagatagcaacagtgattgatgataatactgtacttcatcttacttgccacaaaataacattttataaatcctcaaagtaaaattgagaaatctttaagtttttttcaagtaacataatctatctttgtataattcatatttgggaatatggcttttaataatgttcttcccacaaataatcatgcttttttcctatggttacagcattaaactctattttaagttgtttttgaactttattgttttgttatttaagtttatgttatttataaaaaaaaaaccttaataagctgtatctgtttcatatgcttttaattttaaaggaataacaaaactgtctggctcaacggcaagtttccctcccttttctgactgacactaagtctagcacacagcacttgggccagcaaatcctggaaggcagacaaaaataagagcctgaagcaatgcttacaatagatgtctcacacagaacaatacaaatatgtaaaaaatctttcaccacatattcttgccaattaattggatcatataagtaaaatcattacaaatataagtatttacaggattttaaagttagaatatatttgaatgcatgggtagaaaatatcatattttaaaactatgtatatttaaatttagtaattttctaatctctagaaatctctgctgttcaaaaggtggcagcactgaaagttgttttcctgttagatggcaagagcacaatgcccaaaatagaagatgcagttaagaataaggggccctgaatgtcatgaaggcttgaggtcagcctacagataacaggattattacaaggatgaatttccacttcaaaagtctttcattggcagatcttggtagcactttatatgttcaccaatgggaggtcaatatttatctaatttaaaaggtatgctaaccactgtggttttaatttcaaaatatttgtcattcaagtccctttacataaatagtatttggtaatacatttatagatgagagttatatgaaaaggctaggtcaacaaaaacaatagattcatttaattttcctgtggttgacctatacgaccaggatgtagaaaactagaaagaactgcccttcctcagatatactcttgggagagagcatgaatggtattctgaactatcacctgattcaaggactttgctagctaggttttgaggtcaggcttcagtaactgtagtcttgtgagcatattgagggcagaggaggacttagtttttcatatgtgtttccttagtgcctagcagactatctgttcataatcagttttcagtgtgaattcactgaatgtttatagacaaaagaaaatacacactaaaactaatcttcattttaaaagggtaaaacatgactatacagaaatttaaatagaaatagtgtatatacatataaaatacaagctatgttaggaccaaatgctctttgtctatggagttatacttccatcaaattacatagcaatgctgaattaggcaaaaccaacatttagtggtaaatccattcctggtagtataagtcacctaaaaaagacttctagaaatatgtactttaattatttgtttttctcctatttttaaatttattatgcaaattttagaaaataaaatttgctctagttacacacctttagaattctagaatattaaaactgtaaggggcctccatccctcttactcatttgtagtctaggaaattgagattttgatacacctaaggtcacgcagctgggtagatatacagctgtcacaagagtctagatcagttagcacatgctttctactcttcgattattagtattattagctaatggtctttggcatgtttttgttttttatttctgttgagatatagcctttacatttgtacacaaatgtgactatgtcttggcaatgcacttcatacacaatgactaatctatactgtgatgatttgactcaaaaggagaaaagaaattatgtagttttcaattctgattcctattcaccttttgtttatgaatggaaagctttgtgcaaaatatacatataagcagagtaagccttttaaaaatgttctttgaaagataaaattaaatacatgagtttctaacaattaga

(19) United States
(12) Patent Application Publication
(10) Pub. No.: US 2015/0126433 A1
(43) Pub. Date: May 7, 2015

(54) GROWTH FACTORS FOR THE TREATMENT OF MYCOBACTERIAL INFECTION

(71) Applicant: University of Cincinnati, Cincinnati, OH (US)

(72) Inventors: Francis X. McCormack, Terrace Park, OH (US); Rajamouli Pasula, Mason, OH (US)

(21) Appl. No.: 14/396,925

(22) PCT Filed: Apr. 25, 2013
(86) PCT No.: PCT/US2013/038206

§ 371 Date: Oct. 24, 2014

Related U.S. Application Data
(60) Provisional application No. 61/638,149, filed on Apr. 25, 2012.

(51) Int. Cl. A61K 38/18; A61K 45/06; C07K 14/475

(52) U.S. Cl. CPC A61K 38/18; C07K 14/475; A61K 45/06
USPC 514/2.4

(57) ABSTRACT

Described herein are novel methods and kits for treating mycobacterium infections with KGF. The methods include administering an amount of KGF effective to treat the mycobacterium infection. The mycobacterium may be M. tuberculosis, drug resistant strains of M. tuberculosis, or atypical mycobacterium. The infections may be pulmonary infections. The method and kit may further include additional antimicrobial compounds effective against mycobacterium infections.

### RELATED APPLICATION

The Present application claims priority to U.S. Ser. No. 61/638,149 filed Apr. 25, 2012, the disclosure of which is hereby incorporated herein by reference in its entirety.

### FIELD

The present invention is directed to methods of treating mycobacterium infections, and more particularly, to methods of treating mycobacterium infections with a growth factor.

### BACKGROUND

Mycobacterium tuberculosis (M. tuberculosis) infects one third of the world's population and results in up to 2.0 million deaths per year. Strains resistant to all major anti-tuberculosis drugs have emerged and novel approaches to therapy are needed. Inhaled M. tuberculosis bacteria impact on the airway epithelial lining layer and are internalized into the phagosomes of alveolar macrophages. Infected alveolar macrophages secrete TNFα and chemokines that coordinate the recruitment of T lymphocytes into lung granulomas that function to contain the infected cells and to facilitate the execution of microbicidal programs. T lymphocytes within the lesions interact with alveolar macrophages and secrete IFN-γ, GM-CSF and other cytokines that promote effective intracellular killing by alveolar macrophages through both oxidative and non-oxidative mechanisms. Fusion of the macrophage phagosome with the lysosome, a tightly regulated event that exposes the bacterium to the acidic pH and digestive enzyme milieu within the lysosome, is required for killing. M. tuberculosis can subvert host innate immune responses and survives within macrophages through a variety of adaptive mechanisms, including scavenging iron and inhibiting acidification of the phagosome such as by preventing the fusion of the phagosome with a lysosome, leading to progressive pulmonary infection or latency, the latter with recrudescence years after the primary exposure. Treatment strategies focused on activating or reactivating the microbicidal activities of the alveolar macrophages, including phagosome-lysosome fusion may circumvent the mechanisms of resistance presently employed by M. tuberculosis.

Keratinocyte growth factor (KGF) is a potent epithelial mitogen that is known to contribute to epithelial repair in several organs. KGF is expressed by mesenchymal cells and binds to FGFR2b receptors that are almost exclusively restricted to the epithelium. Previous studies indicate that KGF protects the lung from various insults such as hypoxia, acid instillation and bleomycin and enhances host survival following Pseudomonas aeruginosa-induced lung injury in vivo. In addition, it has also been reported that exogenous KGF results in activation of alveolar macrophages and enhanced clearance of Gram-negative bacteria from murine lungs at least in part by inducing the secretion of GM-CSF from the pulmonary epithelium, engagement of the GM-CSF receptor on alveolar macrophages, and activation of STATS signaling pathway in the phagocyte. The most common serious gram negative infections in the lung are those due to Klebsiella pneumonia, Hemophilus influenza, Pseudomonas aeruginosa, Enterobacter cloacae, and Eschericha coli. These organisms typically produce acute and often life threatening pneumonias that require hospitalization, treatment with IV antibiotics and, in the most serious cases, ICU care and mechanical ventilation. Patients who respond to therapy can usually be discharged within 7-10 days and require an additional three weeks or so to recover. The most common drugs used to treat serious gram negative pulmonary infections in the lung are beta lactamase resistant penicillins such as pipercillin, aminoglycosides such as gentamycin, and third generation cephalosporins such as cefapime. None of these drugs are effective in treating M. tuberculosis infections.

The route and course of infection by M. tuberculosis is distinct from that of infections with Gram negative bacteria. M. tuberculosis infections most commonly produce an initial subclinical or minor clinical illness followed by development of latency, in which the organisms survive within host alveolar macrophages. Reactivation of dormant organisms, often during a period of debility or poor nutrition, can result in chronic, progressive and sometimes life threatening pulmonary infection. The drugs used to treat M. tuberculosis pulmonary infections such as isoniazid, rifampin, ethambutol, and pyrazinamide are not effective against Gram negative pulmonary infections, underscoring the difference between pulmonary infections with M. tuberculosis and Gram negative bacteria. Treatment for M. tuberculosis may be complicated by the development of resistance to the arsenal of drugs presently available to treat the infections, which directly target M. tuberculosis. New methods of treating mycobacterium infections are needed.

### SUMMARY

While the invention will be described in connection with certain embodiments, it will be understood that the invention is not limited to these embodiments. On the contrary, the invention includes all alternatives, modifications and equivalents as may be included within the spirit and scope of the present invention.

Aspects of the invention are directed to methods and kits for treating infections with mycobacterium in a subject by administering KGF to the subject in an amount effective to treat the infection. In particular, aspects of the invention are directed to treating pulmonary infections with mycobacterium. Without being bound to any particular theory, mycobacterium, such as M. tuberculosis, overcome the normal antimicrobial mechanisms utilized by macrophages such as by preventing the fusion of a mycobacterium containing phagosome with a lysosome. KGF induces alveolar macrophage to overcome the mycobacterium induced inhibition of the fusion of the phagosome with the lysosome thus allowing the macrophage's natural killing mechanism to function. This strategy enhances the ability of macrophages, in particular alveolar macrophages to kill the mycobacterium, such as M. tuberculosis, and may also augment other chemotherapeutic approaches to mycobacterium that are resistant to treatment with drugs that directly target the organism. This KGF mechanism is also useful for the treatment of atypical mycobacterial infections, especially those infections that remain uncured despite prolonged three drug therapy. Exemplary atypical mycobacterium are M. avium, M. kanasii, M. abscessus, M. chelonae, M. fortuitum, M. genavense, M. gordonae, M. haemophilum, M. immunogenum, M. malmoense, M. marinum, M. mucogenicum, M. nonchromogenicum, M. scrofulaceum, M. simiae, M. smegmatis, M. szulgai, M. terrae, M. ulcerans, M. xenopi, and combinations thereof.

Thus, an aspect of the invention is directed to methods of treating mycobacterial infections, in particular, pulmonary infections with M. tuberculosis, drug resistant M. tuberculosis, and atypical mycobacterium infections by administering to the subject an amount of KGF effective to treat the mycobacterium infection. KGF may be administered as a standalone therapy or in combination with other antimicrobial agents.

Another aspect of the invention is directed to kits for treating a mycobacterium infection with the kit including a plurality of doses of KGF effective to treat the mycobacterium infection.

### BRIEF DESCRIPTION OF THE DRAWINGS

The accompanying figures, which are incorporated in and constitute a part of this specification, illustrate various embodiments of the invention and, together with a general description of the invention given above and the detailed description of the embodiments given below, serve to explain the embodiments of the invention.

FIG. 1 is graph of data demonstrating that KGF treatment of MLE-15 cells results in a decrease in M. tuberculosis burden in co-cultured RAW 264.7 cells.

FIG. 2 is a graph of data demonstrating that KGF treatment of MLE-15 cells induces production GM-CSF.

FIG. 3 is a graph of data demonstrating that conditioned media from KGF-treated MLE15 cells reduces the growth of M. tuberculosis in RAW 462.7 cells by mechanism partially dependent GM-CSF.

FIG. 4 is a graph of data demonstrating that KGF treatment increases fusion of lysosomes with M. tuberculosis bearing phagosomes in alveolar macrophages.

FIG. 5 is a graph of data demonstrating that KGF treatment improves the body weight of mice infected with M. tuberculosis.

FIG. 6 is a graph of data demonstrating that KGF treatment decreases the colony forming units of M. tuberculosis in lungs harvested of mice infected with M. tuberculosis.

FIG. 7 is a graph of data demonstrating that KGF treatment results in weight gain in mice infected with M. tuberculosis.

FIG. 8 is a graph of data demonstrating that KGF treatment enhances M. tuberculosis clearance from lungs of mice infected with M. tuberculosis.

FIG. 9 is a graph of data demonstrating that KGF treatment enhances the survival of mice inoculated with a high dose of M. tuberculosis.

FIG. 10 is a graph of data demonstrating that KGF treatment enhances clearance of M. tuberculosis in mice after low dose inoculation.

FIG. 11 is a graph of data demonstrating that KGF treatment reduces the burden of M. avium instilled into the lungs of C57B16 mice.

### DETAILED DESCRIPTION

An aspect of the invention is directed to methods of treating an infection with mycobacterium in a subject that includes administering to the subject an amount of a KGF effective to treat the mycobacterium infection.

Forms of KGF which induce the clearance of mycobacterium infections as described herein may be used. For example, in one embodiment, the KGF is a naturally occurring KGF isolated from a biological source using routine methods known in the art, such as by using standard chromatography techniques to isolate KGF from biological tissues. In an alternative embodiment, the KGF is a recombinant KGF having an amino acid sequence as disclosed in SEQ ID NO: 1. The recombinant KGF may also be modified so as to have a shortened amino acid sequence. In an embodiment, the recombinant KGF has an amino acid sequence that corresponds with amino acid residues 32 to 194 inclusive of SEQ ID NO: 1. In another embodiment, the recombinant KGF has an amino acid sequence that corresponds with amino acid residues 55 to 194 inclusive of SEQ ID NO: 1, which excludes an N-terminal region of the protein to improve the stability of the resulting truncated protein. An exemplary pharmaceutically acceptable and FDA approved form of truncated recombinant KGF is palifermin, which is a water-soluble, 140 amino acid protein with a molecular weight of 16.3 kilodaltons. Palifermin is marketed by Amgen Inc. under the brand name Kepivance™, which is supplied as a sterile, white, preservative-free, powder for intravenous injection after reconstitution. When reconstituted in 1.2 ml water, the solution includes 6.25 mg palifermin (5 mg/ml), 50 mg mannitol, 25 mg sucrose, 1.94 mg L-histidine, and 0.13 mg Polysorbate 20 (0.01% w/v).

Recombinant KGF may be produced using standard molecular biology techniques with the nucleic acid of SEQ ID NO: 2. Truncated forms of KGF, such as describe above, may be produced by truncating SEQ ID NO: 2 using standard molecular biology techniques. For example, the nucleic acid sequence of SEQ ID NO: 2 may be amplified by PCR. Truncated forms of KGF may be amplified by selecting primers that amplifies just the desired region of SEQ ID NO: 2 to result in the truncated protein. The amplified DNA is then be ligated into an expression vector between known restriction sites to form a plasmid. The plasmid is cloned into a host cell such as with a standard electroporation transformation procedure. Transformed clones containing the plasmid are selected and allowed to grow under conditions to maximize expression of recombinant KGF. KGF is then isolated and purified to pharmaceutically acceptable level, such as by standard chromatography procedures.

KGF is administered to the subject at a dose effective to treat the mycobacterium infection. In an embodiment, the mycobacterium is M. tuberculosis and strains of M. tuberculosis resistant to other antimicrobial agents. In another embodiment, the mycobacterium is an atypical mycobacterium. Exemplary atypical mycobacterium are M. avium, M kanasii, M. abscessus, M. chelonae, M. fortuitum, M. genavense, M. gordonae, M. haemophilum, M. immunogenum, M. malmoense, M. marinum, M. mucogenicum, M. nonchromogenicum, M. scrofulaceum, M. simiae, M. smegmatis, M. szulgai, M. terrae, M. ulcerans, and M. xenopi.

The effective dose is a dose of KGF that is sufficient to result in an improved clinical outcome for the subject. In an embodiment, an improved clinical outcome may be determined by examining the level of mycobacterium in a sample from the subject. For example, a sample of tissue or fluid such as sputum may be collected from the subject and analyzed for the presence or absence of mycobacterium. The level of mycobacterium may be quantified such as by measuring the colony forming units in the sample per unit sample volume with routine methods. In some embodiments, samples are collected before and after starting treatment and the level of mycobacterium in the samples may then be compared to evaluate the effectiveness of the treatment. In an embodiment, a reduction in colony forming units by half in the sample from the post starting treatment as compared to the pretreatment sample is indicative of treatment with an effective amount of KGF. In another embodiment, the level of mycobacterium in the sample may be compared to a predetermined threshold level established as determinative of an improved clinical outcome. In an embodiment, an improved clinical outcome results in the absence of mycobacterium in the sample or the absence of signs of active infection in image analysis or with biomarkers of infection. Other techniques for evaluating clinical outcome may be employed. For example, x-ray analysis could be used to identify improvement in the image of the lung to indicate the effectiveness of the treatment.

In an embodiment, the effective daily dose is in a range from about 0.1 μg/kg bodyweight to about 10 mg/kg bodyweight. In an alternative embodiment, the daily dose is in the range from about 0.1 μg/kg bodyweight to about 1 mg/kg bodyweight. In a further alternative embodiment, the daily dose is in the range from 1 mg/kg body weight to about 10 mg/kg bodyweight. In an embodiment, the KGF is administered at a daily dose of about 1.5 mg/kg bodyweight to about 5 mg/kg bodyweight. In another embodiment, KGF is administered at a daily dose of about 10 μg/kg bodyweight to about 100 μg/kg bodyweight.

The effective dose will be administered at a rate and over a period of time to result in the desired improved clinical outcome as described above. In an embodiment, the effective dose of KGF will be administered over a period of time ranging between about 1 week and about 6 weeks. In another embodiment, the period of time is between about 1 week and about 2 weeks. In another embodiment, the period of time is between 1 day and about 1 week. The dose may be administered on a schedule determined through clinical evaluations to result in the desired improved clinical outcome. For example, the schedule may include administration that is daily, every other day, every third day, or weekly.

KGF is generally formulated in a pharmaceutically acceptable carrier for administration to the subject. Pharmaceutically acceptable carriers for proteins are well known in the art and typically include a solvent, such as water, one or more salts, and one or more buffers, osmotic balancing agents, and preservatives. The effective amount of KGF may be administered by at least one of intravenous administration, transmucosally via intranasal administration, or by injection via intraperitoneal injection or intravenous injection and by direct application to the infected tissue such by inhalation of an aerosolized formulation of KGF into the lung. Suitable carriers and their formulations are described in Remington: The Science and Practice of Pharmacy (19th ed.) ed. A. R. Gennaro, Mack Publishing Company, Easton, Pa. 1995. For intravenous administration, an appropriate amount of a pharmaceutically-acceptable salt is used in the formulation to render the formulation isotonic. Examples of the pharmaceutically-acceptable carriers include, but are not limited to, saline, Ringer's solution and dextrose solution. The pH of the solution is in a pharmaceutically acceptable range. One of ordinary skill in the art will appreciate that the exact formulation of the composition may be adjusted for use in a particular route of administration and concentration of composition being administered. For example, persons skilled in the art may choose a particular carrier suitable for introduction to the body by injection or by intranasal administration.

In an alternative embodiment, KGF is administered in combination with at least one additional antimicrobial agent useful for treating a mycobacterium infection. Exemplary additional antimicrobial agents include the first line drugs isoniazid, rifampin, ethambutol, and pyrazinamide, as well as the second line drugs streptomycin, amikacin, kanamycin, capreomycin, viomycin, enviomycin, ciprofloxacin, levofloxacin, moxifloxacin, ethinamide, prothinamide, cycloserin, and terizidone. These additional antimicrobial agents include the active components and their pharmaceutically acceptable salts and solvates.

The additional antimicrobial agent may be administered in combination with KGF by the same route of administration or may be coadministered at the same time or at a different time by the same or a different routes of administration so long as the active ingredients from both the KGF and the additional antimicrobial agent are present in the subject at the same time. For example, KGF may be administered intranasally or by injection, while the additional antimicrobial agent is administered orally or in a second injection at the same time or at a different time.

KGF may be packaged as a kit as KGF alone or in the alternative, in combination with the at least one additional active agent, as a complete or partial course of treatment. For example, if a course of treatment require a daily administrations of a single dose of the pharmaceutically acceptable form of KGF over a course of 14 days, then the kit could include a plurality of doses such as the total 14 doses or a subset of doses (such as 7 day increments) to cover the treatment over this period. The kit may further include instructions for administering the dose as well as any devices needed to administer the dose such as syringes, inhalers, etc.

### Example 1

### Materials and Methods

Animals

C57BL/6 mice were purchased from Jackson Laboratories (Bar Harbor, Me.) and housed under pathogen-free conditions in the animal facility of the University of Cincinnati College of Medicine. Mice were given ad libitum access to sterilized food and water. M. tuberculosis infected mice were housed in a BSL3 animal facility. All animal procedures were approved by the University of Cincinnati Institutional Animal Care and Use Committee.

Culture and Isolation of M. tuberculosis

The virulent Erdman strain of M. tuberculosis was obtained from the ATCC (ATCC 35801). The GFP-expressing virulent H₃₇Rᵥ M. tuberculosis strain used for phagolysosome fusion experiments was a gift. M. tuberculosis was cultivated on 7H11 agar plates and harvested in RPMI1640 containing 10 mM HEPES. To minimize problems with clumping, suspensions were gently sonicated in a GenProbe bath sonicator. The accuracy of counts was confirmed by serial dilution and determination of colony forming units (CFUs) on agar plates. To assess M. tuberculosis growth from the infected mice, the lungs were removed aseptically at specified time points, cut into small pieces, and homogenized. Viable M. tuberculosis in the lung tissue homogenates were quantified by serial dilution, plating in duplicate onto 7H11 Middlebrook agar in 6-well plates, incubation at 37° C. in a 5% CO₂ incubator for three weeks, and counting of CFUs. The plates were then incubated for an additional two weeks to detect any slower growing M. tuberculosis species. The results are expressed as mean±standard error mean (SEM) of lung CFUs for each experimental condition.

Cultured Cells

MLE-15 cells are an immortalized cell line derived from the lung tumors of transgenic mice expressing the simian virus 40 (SV40) large T antigen under the transcriptional control of the human surfactant protein C(SP-C) promoter. MLE-15 cells have many characteristics of alveolar type II cells, including epithelial cell morphology, microvilli, cytoplasmic multivesicular bodies, multilamellar inclusion bodies, expression of SP-A, SP-B, and SP-C, and secretion of phospholipids. MLE-15 cells were grown in RPMI 1640 (Gibco BRL) containing 2% fetal bovine serum, 0.5% insulin-transferrin-sodium selenite (Sigma), 5 mg/liter transferrin, 10 mM HEPES, 10⁻⁸ M B-estradiol, and 10⁻⁸ M hydrocortisone. RAW 264.7 (RAW) cells (American Type Culture Collection, Manassas, Va., USA) are a mouse macrophage cell line that was originally established from a tumor induced by the Abelson murine leukemia virus. EGFP- and mCherry-fused actin labeled RAW 264.7 cell lines were also utilized to facilitate discrimination of macrophages from epithelial cells. The RAW 264.7 cell lines were maintained in DMEM (Invitrogen, Burlington, ON, Canada) supplemented with 10% heat-inactivated FBS (Invitrogen, Burlington, ON, Canada) and cultured at 37° C. in a 5% CO₂ atmosphere. MLE-15 cells were plated at 25% confluency in the lower well of transwell plates and were pretreated with KGF (100 ng/ml) or PBS in DMEM (Gibco) overnight. RAW 264.7 cells were seeded at 25% confluency in the upper well of transwell dishes. On the third day, when the RAW 264.7 cells were approximately 50-60% confluent, they were incubated with M. tuberculosis at an MOI of 1:10 for 2 hours, washed and further incubated in co-cultures with MLE-15 cells. After 5 days, the RAW 264.7 cells were harvested, lysed, and plated onto 7H11 Middlebrook agar plates. CFUs were quantified as outlined above. GM-CSF concentrations were measured in cell-free supernatants of culture samples using a commercially available ELISA kit (R&D System, Minneapolis, Minn.) according to the manufacturer's instructions and were expressed as pg/ml of the culture supernatant.

Isolation of Murine Alveolar Macrophages

Mice were sacrificed and bronchoalveolar lavage was performed by 10 cycles of instillation and aspiration of 1 ml Hanks' balanced salt solution (HBSS) with 0.6 mM EDTA. The BAL fluid was centrifuged at 1500×g for 5 min, and the cells were re-suspended in lysis buffer containing 0.15 M NH₄Cl with 0.01 M KHCO₃, washed, and re-suspended in 0.9% NaCl. Cell counts were determined using a hemocytometer and cell differentials were determined by examination of cytopreparation smears (Cytospin II, Shandon Southern Instruments, Inc., Sewickley, Pa.) after processing with the Hema 3 staining system (Fisher Scientific Inc, Pittsburg, Pa.). Viability of alveolar macrophages was determined by Trypan blue exclusion.

Infection of Mice with M. tuberculosis

Mice were infected i.n. with Erdman M. tuberculosis (2.5×10¹-1×10⁶) suspended in 50 p. 1 HBSS in a class III biohazard safety cabinet and maintained in the BSL3 animal facility at the University of Cincinnati. Mice were infected i.n. with Erdman M. tuberculosis. Mice were treated with i.n. PBS or KGF (5 mg/kg) as indicated and either monitored for weight change and vital status, or sacrificed for the determination of CFUs in lung homogenates.

Assessment of Phagosome-Lysosome Fusion

To examine the co-localization of M. tb-containing phagosomes with LAMP-1 antibody, alveolar macrophages (AMs) from KGF- or saline-treated mice were plated onto slides at 1×10⁵ AMs/well in DMEM for 4 hours and incubated with EGFP-M. tuberculosis H₃₇Rᵥ at a multiplicity of infection (MOI) of 10:1 for 2 hours. After washing the monolayer, the AMs were fixed with 1% paraformaldehyde for 10 min and with 2% paraformaldehyde for an additional 10 min. The fixed monolayers were permeabilized with 100% methanol for 5 min, washed 3 times with Dulbecco PBS, and blocked overnight at 4° C. with PBS containing normal goat serum, 10% heat-inactivated FCS (Hyclone) and 5 mg/ml BSA. After washing, the monolayers were incubated with mouse anti-human LAMP-1 antibody (University of Iowa Hybridoma Facility, Iowa City, Iowa) for 1 hour, followed by incubation with mouse IgG conjugated with Alexa Flour 555, (1:5000 dilution) (Molecular probes). After washing, the slides were separated from the wells, overlaid with a coverslip with mounting media and sealed with nail polish. The slides were viewed using a confocal microscope (Olympus FV1000). Microscopic fields were selected at random to view M. tb-containing phagosomes.

Statistical Analysis

All experiments were conducted in triplicate with a minimum of 3 experiments. The results were expressed as means±SEM. The statistical difference between the control and experimental data was determined by Student's t test or ANOVA with paired comparisons. A p value <0.05 was considered to be statistically significant.

Results

Co-Culture Models of Alveolar Epithelial Cells and Macrophages Recapitulate GM-CSF-Dependent Killing of M. tuberculosis, In Vitro

Previous reports indicate that KGF enhances pulmonary epithelial GM-CSF production, which in turn activates macrophages. To determine if GM-CSF dependent AM activation contribute to the mechanism of enhanced M. tuberculosis clearance in KGF-treated mice, a series of in vitro co-culture experiments were conducted.

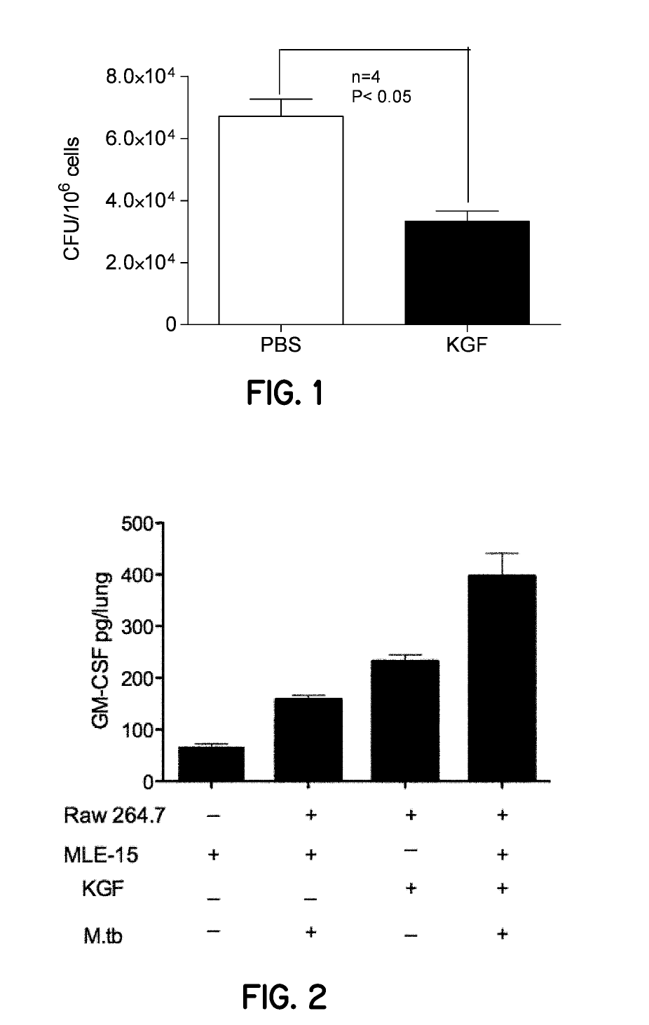

MLE-15 cells were plated in the lower well of transwell plates and pretreated with KGF (100 ng/ml) or vehicle (PBS) overnight. RAW 264.7 cells were infected with M. tuberculosis at an MOI of 10:1, washed and added to the upper well. After 5 days of co-culture, the RAW 264.7 cells were harvested, lysed, plated onto 7H11 Middlebrook agar plates and M. tuberculosis CFUs were quantified. The data demonstrate that KGF treatment of MLE-15 monolayers reduced the growth of M. tuberculosis contained within co-cultured AMs approximately 2.5 fold (p<0.05) (FIG. 1). The GM-CSF contents of the media supernatant under various culture conditions are shown in FIG. 2. These data indicate that MLE-15 cells produce a small amount of GM-CSF, which is augmented 3-4 fold when either KGF is added, or when co-cultured with AMs. When M. tuberculosis infected RAW 264.7 cells are co-cultured with KGF-treated MLE-15 cell monolayers, there is an approximately 7-fold increase in the level of GM-CSF in culture media. Conditioned media from MLE-15 cells treated with KGF (KGF-conditioned medium) was then used to determine if GM-CSF was the factor responsible for the enhanced killing of M. tuberculosis as shown in FIG. 3. Incubation of M. tuberculosis infected RAW 264.7 cells with conditioned medium significantly reduced the growth of intracellular M. tuberculosis about two fold. The effect of KGF-CM was partially by a neutralizing antibody to GM-CSF but not by an isotype control antibody. These results indicate suggest that KGF treatment of MLE-15 cells induces GM-CSF production which contributes to the decrease in M. tuberculosis burden in co-cultured RAW 264.7. However, since the GM-CFS antibody failed to completely block the KGF effect on CFU, these data also indicate that KGF is acting through some mechanisms that are independent of the GM-CSF pathway. These data support a dual for KGF activity that is partially acting through the GM-CSF pathway as well as through an independent pathway to induce the antimicrobial activation of macrophages.

Effect of KGF on Phagosome-Lysosome Fusion in M. Tuberculosis-Infected Alveolarmacrophages

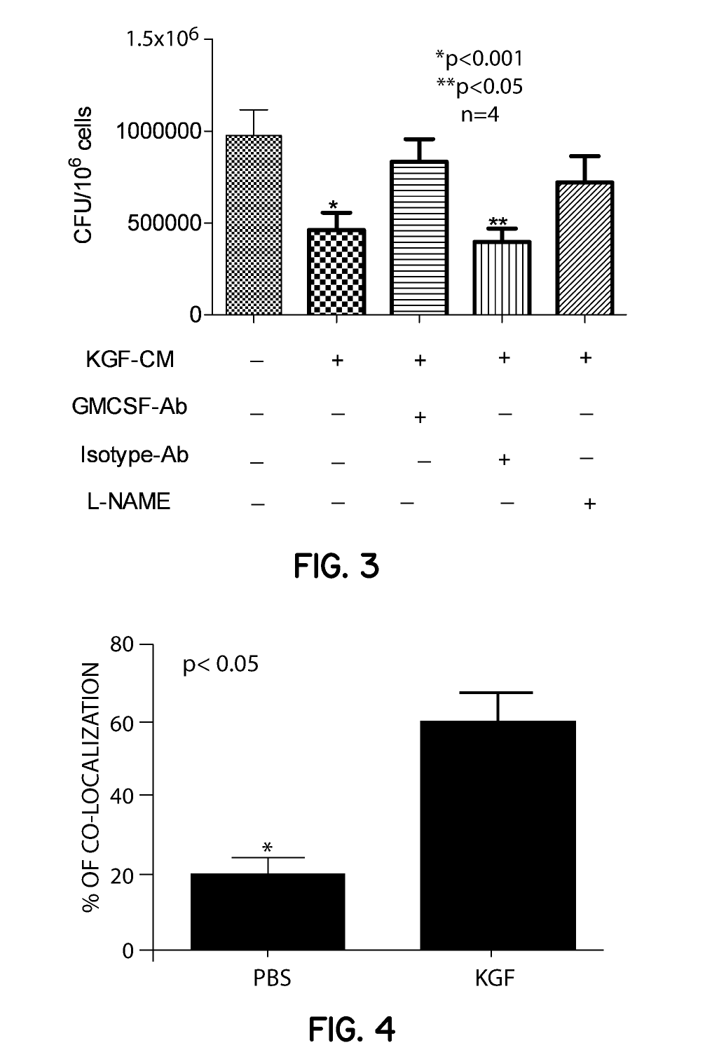

In order to assess the mechanism by which KGF decreases M. tuberculosis growth in macrophages, the effect of KGF on phagosome-lysosome fusion in M. tuberculosis infected AMs was examined. AMs were isolated from KGF or PBS pre-treated animals and incubated with EGFP expressing M. tuberculosis. Fusion of phagosomes with lysosomes was detected by staining the late endosomal/lysosomal compartment with LAMP-1 antibody and examining the cells by confocal microscopy to assess the degree of phagosome-lysosome fusion. AMs isolated from KGF-treated mice showed significantly increased co-localization of EGFP-M. tuberculosis with LAMP-1 compared to PBS controls (FIG. 4). The percentage of co-localized EGFP-M. tuberculosis was 28.9±3.8% in KGF-treated mouse AMs compared to 10.9±1.3 in PBS control AMs (p<0.005) (FIG. 4). These data suggest that KGF pre-exposure to mice results in increased phagosome-lysosome fusion in M. tuberculosis-infected AMs.

Infection by M. Tuberculosis Results in Inoculum Size-Dependent Mortality in C57BL/6 Mice

Experiments were performed to determine the optimal M. tuberculosis inoculum for subsequent experiments. Mice received Erdman M. tuberculosis i.n. at doses ranging from 10¹ to 10⁹ organisms and were observed daily for up to 35 weeks. Mice that received 10⁹ and 10⁸ CFU of M. tuberculosis survived for up to 8 and 12 weeks respectively, while those that received 1×10⁴-1×10²M. tuberculosis survived beyond 32 weeks. Based on these data, we chose an inoculum of 10⁵ organisms per mouse for most experiments, which as close of the LC50 of 4.8×10⁴ organisms. These results indicate that M. tuberculosis (Erdman) strain exhibits dose dependent mortality in C57BL/6 mice.

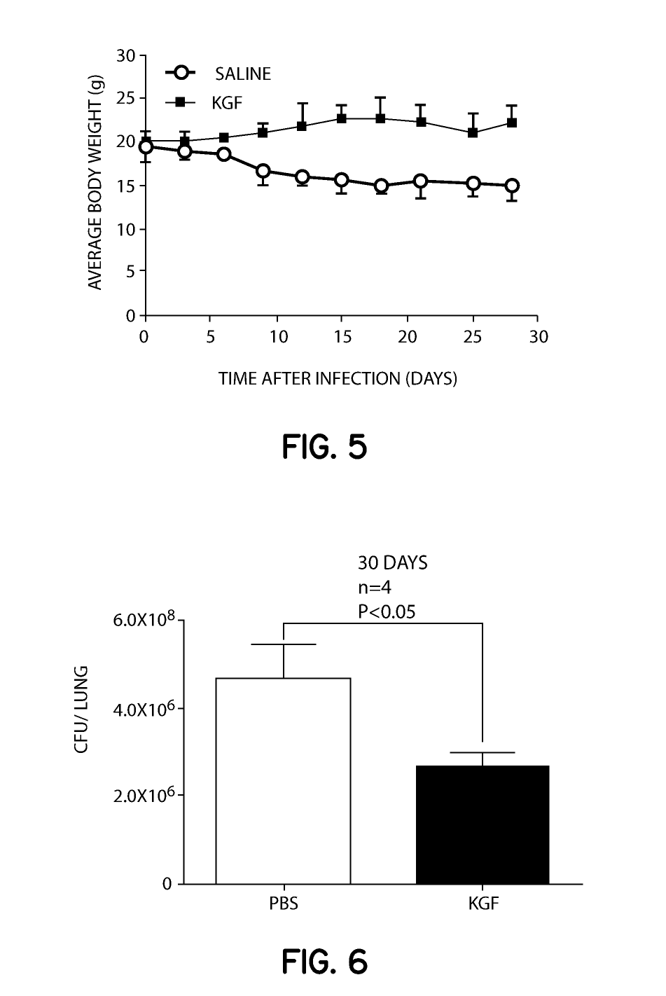

A single pre-infection dose of KGF protects mice from weight loss and enhances clearance of M. tuberculosis from mouse lungs—The effect of KGF pretreatment on body weight and pulmonary burden of M. tuberculosis bacilli was assessed. Mice were treated with a single i.n. dose of KGF (5 mg/kg) or PBS, and then inoculated i.n. with 10⁵ M. tuberculosis 24 hrs later. The mice that received PBS steadily lost about 30±0.55% of their baseline body weight by day 20, but the mice that received KGF gained weight and body mass20±0.25% above baseline by day 20 (n=4, p<0.05) (FIG. 5). At day 30, the lungs were harvested and CFUs in the lung homogenates were quantified. KGF pretreatment resulted in a 1.9±0.25 fold reduction in the number of CFUs in the lung of KGF-treated mice compared to the PBS-treated controls (p<0.05, FIG. 6). These data indicate that prophylactic administration of KGF enhances the clearance of M. tuberculosis from the lungs and reverses the loss of body weight. The data are expressed as mean±SEM.

Serial Administration of KGF Attenuates Murine Pulmonary M. Tuberculosis Infection

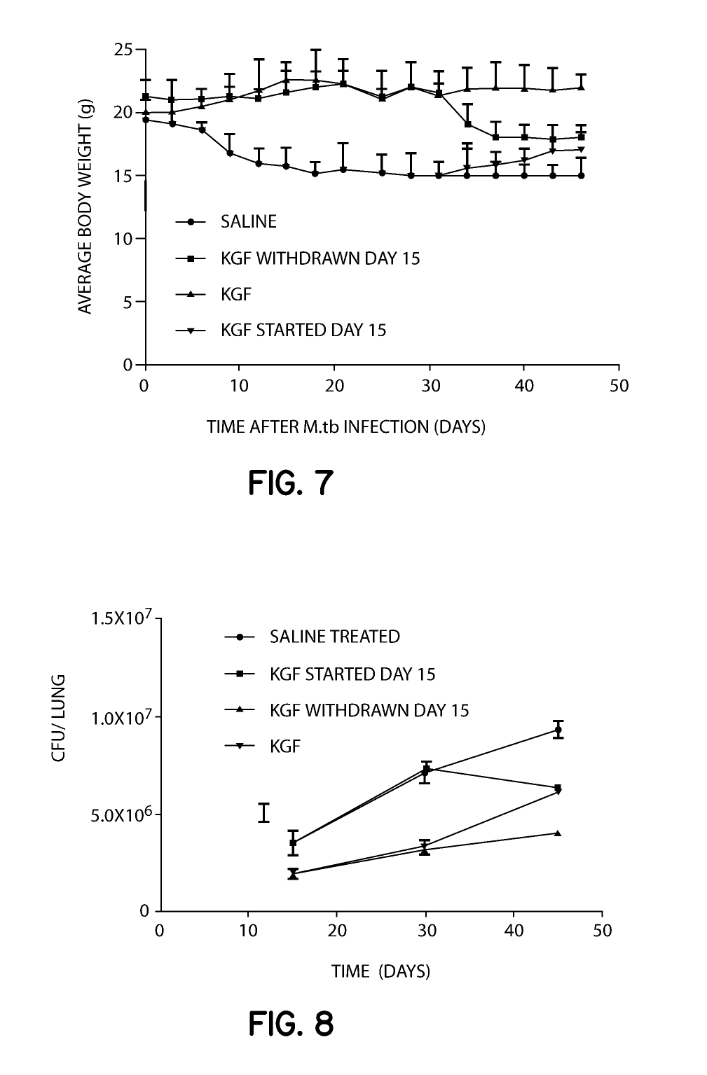

To determine if serial pretreatment with KGF protects mice from weight loss and progressive mycobacterial infection, C57B16 mice were treated with KGF (5 mg/kg) or PBS i.n. and then inoculated i.n. with 10⁵ M. tuberculosis 24 hours later and then serially treated with KGF or PBS every 3 days for 45 days (FIG. 7). During this period, the mice that received PBS lost approximately 30% of their weight. In contrast, the mice that received KGF gained weight to 22.24±0.56% above their baseline body weight. The difference in body weight in the two groups was significant at day 20 (p<0.001) and day 45 (p<0.001). In mice that received only PBS, the growth of M. tuberculosis in lung homogenates increased by approximately 3 fold over the 45 days, whereas in the KGF-treated group there was a marked decrease in the amount of growth over this same time period (FIG. 8). These data demonstrate that continuous treatment with KGF for 45 days protects mice from weight loss and reduces the bacterial burden in the lungs.

KGF Treatment of Mice with Established M. Tuberculosis Infection Reverses Weight Loss and Reduces the Bacterial Burden in the Lung

With further attention to FIGS. 7 and 8, to more closely model clinical M. tuberculosis infection, mice with established infection (day 15) were monitored for body weight changes and bacterial burden in the lungs associated with initiation of therapy. Mice were i.n. inoculated with 10⁵ M. tuberculosis, observed without further intervention for 15 days, and then treated with i.n. KGF for an additional 30 days. Over the first 15 days after infection, the body weight fell byl5.6±0.06%. After initiation of KGF on day 15, however, the weight stabilized and began to increase on about day 30 (FIG. 7). By day 45, body weight was restored in the KGF group, and was significantly greater than mean body weight in the group that had received PBS only over 45d (FIG. 7). The bacterial burden continued to rise from day 15 through day 30 despite initiation of KGF on day 15, but decreased somewhat lower by day 45 relative to the PBS group. The day 45 bacilli counts in the mice were significantly greater than those in group that received PBS q3d×45 days, and more than those that received KGF q3d×45d. These data indicated that treatment of established M. tuberculosis infection with KGF reverses weight loss and reduces the burden of M. tuberculosis in the lung.

Resumption of Weight Loss and Progression of M. tuberculosis Infection Following Withdrawal of KGF Treatment

With further attention to FIGS. 7 and 8, experiments were performed to determine the effect of withdrawal of treatment on M. tuberculosis infection. M. tuberculosis infected mice were treated with KGF (5 mg/kg) every third day for 15 days, and then observed without intervention for 30 days. Body weight remained stable through about day 30, and then fell to below the baseline by day 45. The bacterial burden in the lung increased in the interval from day 15 through day 30, and at an accelerated rate in the interval between day 30 and day 45. By day 45, 30 days after withdrawal of treatment, the bacterial burden was significantly greater in the animals that were withdrawn from KGF at day 15 than in the animals that were treated with KGF q3d×45d and significantly less than animals that were treated with PBS q3d×45d (FIG. 8).

KGF Enhances Survival of Mice that are Infected with a Lethal Dose of M. tuberculosis

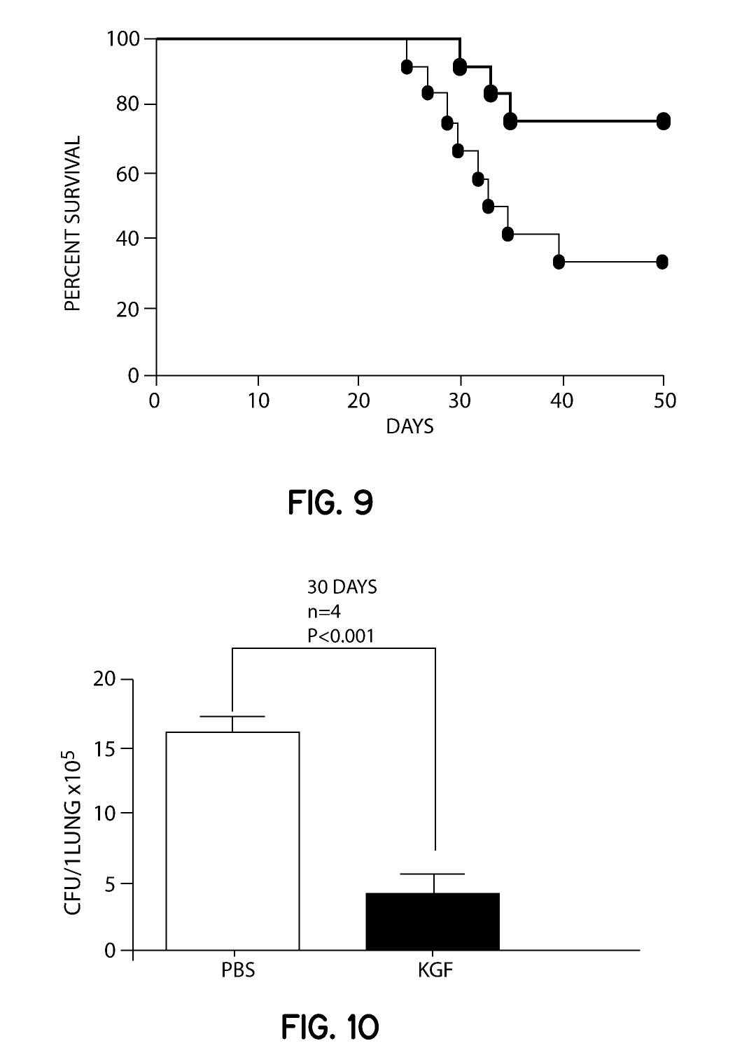

To determine if KGF treatment enhances the survival of mice that are infected with a lethal dose of M. tuberculosis, mice were inoculated with 1×10⁸ Erdman M. tuberculosis, and then treated with i.n. KGF or saline every third day for two weeks. Vital status was monitored on a daily basis over a 50 day period starting from the day of infection (n=9 mice per group). The data demonstrate that 50 day survival was significantly greater in the KGF-treated group than in the PBS-treated group (p<0.05) (FIG. 9). These data indicate that KGF protects mice from a lethal i.n. inoculum of M. tuberculosis.

KGF Enhances the Clearance of M. tuberculosis Inoculated at Doses that More Closely Mimic Inhalational Exposure

Low dose i.n. inoculation of M. tuberculosis more closely mimics the natural infection in humans, but mortality analysis in rodents becomes less practical with this model because of the prolonged time to death. Therefore the effect of KGF on the bacterial burden in the lungs over the course of 30 weeks was measured. Mice were inoculated with 1×10² Erdman M. tuberculosis. After 30 weeks, mice were given i.n. saline or KGF (5 mg/kg) every third day for two weeks. Four weeks after the commencement of the intervention, the lungs were harvested and homogenized, and CFUs were quantified. We found that KGF enhanced the clearance of M. tuberculosis by >5 fold (n=4, P<0.001) (FIG. 10). Results are shown as means±SEM. These data indicate that KGF enhances the clearance of M. tuberculosis in established infection due to a low dose inoculation that mimics inhalational tuberculosis.

The data herein demonstrate that a single dose of KGF administered prior to M. tuberculosis infection or serially post infection protects the animals from weight loss and reduces the burden of M. tuberculosis bacilli in lung tissue. KGF was effective at reversing weight loss and enhancing the pulmonary clearance of M. tuberculosis in established infection, including a low dose inoculum that more closely mimics inhalational exposure, in attenuating mortality from a lethal inoculum of M. tuberculosis. These data also demonstrate that withdrawal of KGF resulted in resumption of weight loss and M. tuberculosis growth. These data indicate that KGF treatment is effective in attenuating M. tuberculosis infection whether delivered as prophylaxis prior to exposure to the bacterium or as treatment for an established infection. These data validate the strategy of administering KGF to enhance alveolar host defense in mycobacterial infections, including those caused by antibiotic-resistant strains.

### Example 2

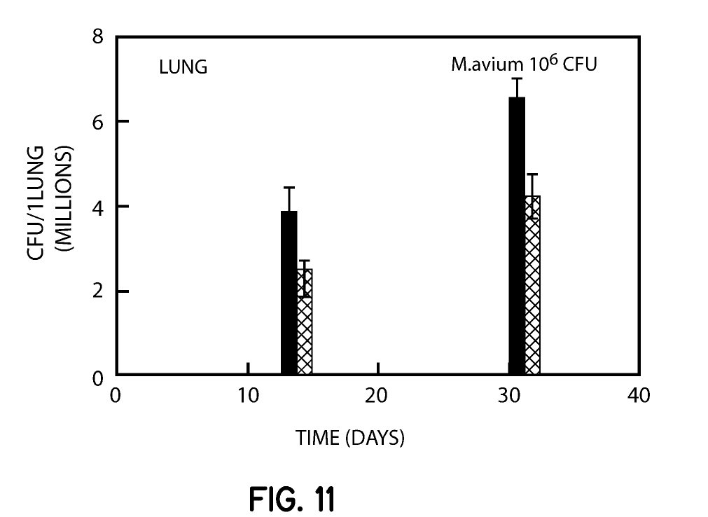

KGF treatment reduces the burden of M. avium, an atypical mycobacterium, instilled into the lungs of C57B16 mice. For this example, mice were treated with 5 mg/kg KGF or saline vehicle and 24 hrs later were inoculated i.n. with M. avium (1×10⁶) suspended in 50 μl PBS. KGF or saline was subsequently administered i.n. every third day for two weeks and then withdrawn. Fifteen or thirty days following M. avium administration, mice were sacrificed and the burden of M. avium was determined from lung homogenates by counting colony forming units on agar plates as described above in Example 1. As demonstrated by the data presented in FIG. 11, the colony forming units from lung homogenates was decreased at both the 15 day and 30 day time points suggesting that KGF treatment is effective at treating infections with atypical mycobacterium.

While the present invention has been illustrated by the description of specific embodiments thereof, and while the embodiments have been described in considerable detail, it is not intended to restrict or in any way limit the scope of the appended claims to such detail. The various features discussed herein may be used alone or in any combination. Additional advantages and modifications will readily appear to those skilled in the art. The invention in its broader aspects is therefore not limited to the specific details, representative apparatus and methods and illustrative examples shown and described. Accordingly, departures may be made from such details without departing from the scope or spirit of the general inventive concept.

1. A method of treating an infection with mycobacterium in a subject comprising administering to the subject an amount of a Keratinocyte Growth Factor (“KGF”) effective to treat the mycobacterium infection.

2. The method of claim 1 wherein the KGF is a recombinant KGF.

3. The method of claim 2 wherein the recombinant KGF is palifermin.

4. The method of claim 1 wherein the KGF has an amino acid sequence consisting of SEQ ID NO: 1.

5. The method of claim 4 wherein the KGF has an amino acid sequence consisting of amino acid residues 32 to 194 inclusive of SEQ ID NO: 1.

6. The method of claim 4 wherein the KGF has an amino acid sequence consisting of amino acid residues 55 to 194 inclusive of SEQ ID NO: 1.

7. The method of claim 1 wherein the KGF has an amino acid sequence encoded by the nucleic acid sequence consisting of SEQ ID NO 2.

8. The method of claim 1 wherein the mycobacterium is selected from the group consisting of M. tuberculosis, M. avium, M. kanasii, M. abscessus, M. chelonae, M. fortuitum, M. genavense, M. gordonae, M. haemophilum, M. immunogenum, M. malmoense, M. marinum, M. mucogenicum, M. nonchromogenicum, M. scrofulaceum, M. simiae, M. smegmatis, M. szulgai, M. terrae, M. ulcerans, M. xenopi, and combinations thereof.

9. The method of claim 8 wherein the mycobacterium is M. tuberculosis.

10. The method of claim 8 wherein the mycobacterium is M. avium.

11. The method of claim 1 wherein the amount of KGF is administered by at least one of intravenous administration, intranasal administration, or intraperitoneal administration.

12. The method of claim 1 wherein the KGF is administered for a period of time sufficient so that the mycobacterium is no longer detectable in the infected tissue.

13. The method of claim 1 wherein the mycobacterium infection is a pulmonary infection.

14. The method of claim 1 further comprising at least one additional antimicrobial agent.

15. The method of claim 14 wherein the at least one additional antimicrobial agent is selected from the group consisting of isoniazid, rifampin, ethambutol, pyrazinamide, streptomycin, amikacin, kanamycin, capreomycin, viomycin, enviomycin, ciprofloxacin, levofloxacin, moxifloxacin, ethinamide, prothinamide, cycloserin, terizidone, and combinations thereof.

16. A kit for treating an infection with a mycobacterium in a subject comprising a plurality of doses of KGF in an amount effective to treat the infection.

17. The kit of claim 16 further comprising a plurality of doses of at least one additional antimicrobial agent in an amount effective to treat the infection in combination with doses of KGF.

18. The kit of claim 17 wherein the at least one additional antimicrobial agent is selected from the group consisting of isoniazid, rifampin, ethambutol, pyrazinamide, streptomycin, amikacin, kanamycin, capreomycin, viomycin, enviomycin, ciprofloxacin, levofloxacin, moxifloxacin, ethinamide, prothinamide, cycloserin, terizidone, and combinations thereof.

19. The kit of claim 16 further comprising a device for administering the plurality of doses of KGF.
